(19) United States
(12) Patent Application Publication
(10) Pub. No.: US 2019/0008604 A1
(43) Pub. Date: Jan. 10, 2019

(54) SYSTEM AND METHOD FOR MARKING AND SUBSEQUENTLY LOCATING SITES OF BIOPSIES USING RFID TECHNOLOGY

(71) Applicant: HINDSITE, LLC, Weston, MA (US)

(72) Inventors: Adam Scott LANDSMAN, Weston, MA (US); Paul Vonryll GRYSKA, Chatham, MA (US); Joseph FERRUCCI, Weston, MA (US)

(73) Assignee: HINDSITE, LLC, Weston, MA (US)

(21) Appl. No.: 16/014,582

(22) Filed: Jun. 21, 2018

Related U.S. Application Data
(60) Provisional application No. 62/529,768, filed on Jul. 7, 2017.

(51) Int. Cl. A61B 90/00; A61B 1/018; A61B 90/98; A61B 1/00

(52) U.S. Cl. CPC A61B 90/39; A61B 1/018; A61B 2090/397; A61B 1/00131; A61B 90/98

(57) ABSTRACT

A system and method for marking sites of biopsied tissues using markers, such as radiopaque microspheres, ink-like stain, and radio-frequency identification chips, delivered to the site of the biopsy by a construct advanced through a port in a colonoscope and locatable by x-ray equipment, a camera attached to a colonoscope, or a radio-frequency identification chip reader.

### CROSS REFERENCE TO RELATED APPLICATION

This application claims the benefit of provisional application No. 62/529,768 filed in the United States Patent and Trademark Office on Jul. 7, 2017, the contents of which are incorporated herein by reference.

### FIELD

This disclosure relates to the field of endoscopy. The system and method described herein are used for marking the site where a polyp or other soft tissue lesion is removed endoscopically from the gastrointestinal tract, for example, by placing a marker in the submucosa of the intestine, but nothing herein shall limit the scope of this invention which can also be used for biopsies of other tissues, such as in the esophagus or in the liver.

### BACKGROUND

During examinations of the intestine, endoscopy is used to visualize the lining of the intestines. Typically when a polyp is encountered, under visualization with an endoscope, the polyp is grasped and removed from the wall of the intestine. It is generally important at this time to mark the location of the removed tissue, so that resection or other medical procedures may be performed at the location of the removal tissue, depending on the results of the biopsy, depending on later, medical recommendations, or pursuant to other follow-up procedures.

Historically, these marks to the intestinal wall have been done using a liquid stain injected into the intestinal wall. This has been viewed as a relatively imprecise method due to the fact that the dye may be released over large areas of the intestinal wall causing excessive streaking rather than placing it within the wall of the intestine. Furthermore, it is possible for the dye to flow to adjacent areas making the biopsy site less precise. Frequently it is difficult to identify and differentiate one marked site from the next. Also, this dye is not radiopaque and therefore may be extremely difficult to find when returning to the patient for further resection, either by colonoscopy or by laparoscopic surgery.

The alternative technique that has been most commonly employed involves deployment of a metallic clip to the mucosa, that is, the wall of the intestine. In many cases, it is desirable for a physician to locate the site of a soft tissue biopsy, especially if needed year after year. However one potential complication of this method is that the clip frequently breaks loose and then the biopsy site is no longer marked. In particular, because clip markers are clamped to the surface of the inner wall of the intestine, they may become dislodged, such as when waste products pass through the intestine. As a result, most clip-on markers may often eventually break free of the inner intestinal wall. Depending on the quality of the tissue the clip is attached to, and the quality of the clip, the marker may be lost within the first week, or may remain attached for several weeks or even months. In many cases, the marker may become lost within the first year. In all events, dislodgement of the marker may interfere or impede subsequent location and re-examination of the site of a soft tissue biopsy, especially if needed year after year. In many cases, it is critical for a physician to locate and re-examine the site of a soft tissue biopsy year after year.

Other prior art techniques for tracking the location of a biopsy may include measuring the distance from the anus to the biopsy site; however this technique has also proved to be highly inaccurate.

One additional consideration is that many times, a biopsy site that is marked during colonoscopy (i.e., internal to the intestine) may have to be revisited laparoscopically (i.e., external to the intestine within the peritoneal cavity). Therefore the marker that is used must be easily found when working from the exterior of the intestinal wall. This may be extremely difficult to do when a portion of the intestine has moved or folded on itself. Consequently, there is a need for an improved marking system and method so that the various biopsy sites can be located during laparoscopic resection.

Accordingly, needs exist for a way to mark a biopsy site or polyp resection site that can be performed simply and reproducibly, typically during colonoscopy, but as well by endoscopy in any part of the gastrointestinal tract, in a manner that allows for subsequent location of the site that is more permanent in nature and for a way to find the place of biopsy marked in this way efficiently and accurately after the elapse of time.

### SUMMARY

In the broadest terms, one device described here is used to firmly grasp the polyp site or biopsy site. Once the tissue is held in place, a needle is advanced into the submucosal layer of the intestinal wall and a small quantity of radiopaque markers are introduced into this layer. This may be accompanied by a small amount of an ink-like stain or dye. By placing these radio opaque markers and stain into the submucosal layer of the intestine, these materials are protected and embedded more permanently.

A critical aspect of the device described here, is the fact that it has been designed to be inserted within the wall of the intestine. In the first embodiment of such device, a construct is passed through one portal of a colonoscope or endoscope. Small jaws are used to firmly grasp the inner wall of the intestine, most likely with a trigger type mechanism. Once the soft tissue is held it can be slightly elevated and a plunger can be used to advance the needle containing the microspheres and stain into the submucosal layer. A second push of the plunger would release the microspheres and stain into the proper layer thereby protecting these markers from further disruption or injury. Once the ink and marker have been dispensed, the jaws are released and the construct can be withdrawn. The captured polyp can be removed, or a second instrument can be introduced to remove the polyp.

Another embodiment of this device involves a snare that is placed around the polyp. Once the polyp is firmly grasped, it is elevated in order to facilitate injection and placement of the microspheres and ink beneath it. The polyp can then be detached from the intestinal wall and the specimen can be withdrawn along with, or separately from the harvest/marking instrumentation. The stain and microspheres are left behind in the intestinal wall submucosa at the site of interest.

In one embodiment of this device, the spheres have radio-frequency identification (“RFID”) capability. The RFID may be placed as a freestanding item, or may be placed in or on the spheres; either would be especially useful for tracking and distinguishing multiple polyp sites and biopsy sites. Another embodiment of this device utilizes collagen or similar coating over the microspheres to facilitate their attachment within the intestinal wall. By using multiple spheres, the position of the spheres relative to each other creates a unique geometric appearance. For example, using three spheres, the spheres may align as a straight line, or in a triangular fashion. The distance between the corners of the triangle will vary, resulting in a unique placement pattern. In this way, one grouping of three or more spheres can be differentiated from other groups of spheres.

A related embodiment is based on the insertion of an RFID chip into the wall of the intestine using a specialized needle as described herein that incorporates a side port to deliver the chip precisely into the correct layer within the wall of the intestine, while avoiding the very dangerous and potentially hazardous complication of intestinal wall perforation. Traditional needles used to deliver dyes to mark polyp locations are on the order of 18-23 gauge diameter. This diameter is too small to allow a 1 mm marker to pass through its lumen. Although one could make a smaller marker, there is a limitation in that it becomes more difficult to find using x-ray means in the case in which a simple radiopaque marker is too small to be detected.

According to another implementation, in order to accommodate an appropriately sized marker without using a larger diameter needle, disclosed herein is a needle that has a small notch along its side, at which the large sized marker can be secured, advanced through the inner wall of the intestine, and deployed by pushing the marker out of the notch before withdrawing the needle. By placing the marker within the tight confines of the wall of the intestine at the intima media, the marker is less prone to move after placement. The advantage of using an RFID is that it can be coded with a serial number unique to that polyp site. The serial number may be a simple two- or three-digit number, or other form of indicia, specific enough so that the marker location of each polyp within a patient is unique, but generalized so as to be consistent with applicable privacy frameworks with regard to patient identification. The RFID chip is identifiable with an RFID reader inserted through an endoscope or through a laparoscope; additionally, an RFID chip can be constructed so as to be radiopaque, so that it could also be located with X-ray devices without intrusion into the body.

In still further implementations, an RFID marker used may be used or devised that has a sufficiently small size and/or sufficiently strong signal (or associated detection) so that use of radiopaque material with such an RFID marker is optional, with the advantage of reducing subsequent exposure of the patient to x-rays. In this implementation, the smaller RFID device may simply be injected directly using existing needle technology, without the need for a specialized needle to dispense larger markers from a side port.

In order to decrease the size of the RFID chip, this disclosure is based on the use of a passive (i.e. non-powered) chip instead of an active marker that requires a power source. Additionally, the RFID chip in another embodiment can be coated with collagen or other coatings in order to facilitate embedding the marker in the intestinal submucosa. The RFID reader of the present disclosure is small enough to fit through standard endoscope ports and shall be constructed to use a proprietary wavelength for reading the codes so that unauthorized readers would not be effective in identification of location or information stored therein.

RFID chips in another embodiment would be delivered simultaneously with radiopaque beads, so that if for some reason the RFID could not be found electronically, the site could still be located by x-ray equipment. An RFID reader in one embodiment would be located outside the body only, and can be fitted out with antenna wires that can be extended through the portal of an endoscope, or can be inserted laparoscopically and extended into the region of the marker to find and identify the desired RFID chip; however, the reader can also be constructed in accordance with the disclosures of this application as a small device that can reside within for example a colonoscope or that can be attached to the end of such a colonoscope that would utilize a much shorter antenna for detecting and locating the RFID chip.

For passive RFID chips, detection works by passing the antenna within a few inches of the chip, which movement can inductively or capacitively excite the chip, which then relays a signal back to the reader. The RFID reader and the RFID marker chip are matched so that the reader can detect the marker. In one embodiment, the reader may use encrypted or proprietary protocols, and is configured so as to be attached to the end of an endoscope with antennae wrapped around or incorporated into the tip of the endoscope. As such, the reader may be custom fabricated for the type and brand of, for example, a colonoscope being used, or may be configured to be used with laparoscopes. Signal strength, frequency, and other parameters associated with RFID/reader pairing, pairs the reader and the marker chip can accommodate suitable to the location, shapes, and environments of the scopes anticipated to be used in the current disclosure.

In one implementation, a system is disclosed for marking locations of biopsied tissues, that is tissues, such as polyps, removed from the body for analysis, such as analysis for potential carcinoma. The system includes a scope having a port in which a biopsy instrument is housed. The biopsy instrument has a disposition that is associated therewith and configured to dispose a marker in the form of an RFID chip in tissue at the site of the biopsy.

The disclosed system includes a reader adapted to detect signals emanating from the possible locations of where RFID chips would potentially be placed in the corresponding medical procedure. Detection can be accomplished by advancing an endoscope or colonoscope through the intestine having at its tip an RFID reader and an antenna wrapped around the end of the scope. The reader is programmed to emit a continuous signal that will excite the RFID marker chip embedded in the intestinal wall. As the RFID reader is advanced towards the marker chip, it excites the chip, signaling its location to the reader. As an alternative, the chip can be replaced with a radiopaque marker like a pellet, and can be located with an x-ray device such as a fluoroscope.

In another implementation, a system is disclosed by which an RFID marker in enclosed in a detachable tip of a needle-like structure that can be delivered to the spot of the biopsy directly.

In still further implementations, the above-described system comprises a system for marking locations where polyps or other soft tissue lesions have been removed endoscopically, so that such removal locations may be more precisely revisited in subsequent endoscopic, laparoscopic, or other targeted medical procedures.

It should be appreciated that the instant disclosure is not to be seen as limited to marking and detection of sites in the colon or intestine, generally. Using the RFID technology described herein, a chip can be implanted in a variety of tissues within the body, including, but not limited to, marking of tissue in patients having Barrett's esophagus and tagging of bleeding lesions in the liver.

### BRIEF DESCRIPTION OF THE DRAWINGS

FIGS. 1A and 1B show the progress of an endoscope of the present disclosure, such as a colonoscope, having an RFID reader, and being inserted into an intestinal lumen and moved closer to a biopsy site marker.

FIG. 2 is a cutaway view of an intestinal lumen.

FIG. 3 is a side view of one embodiment of a device of the present disclosure deployed from the distal end of a colonoscope.

FIG. 4 is a side view of one embodiment of a device of the present disclosure having pierced the mucosa of an intestinal wall.

FIG. 5 is a side view of one embodiment of a device of the present disclosure having dispensed markers into the sub-mucosa of the intestinal wall.

FIG. 6 is a side view of one embodiment of a device of the present disclosure having grasped and elevated a polyp from the intestinal wall.

FIG. 7 is a side view of one embodiment of the device of the present disclosure having lassoed a polyp from the intestinal wall.

FIG. 8 is a side view of a second embodiment of the device of the present disclosure.

FIG. 9 is a side view of a second embodiment of the device of the present disclosure having pierced the mucosa of an intestinal wall.

FIG. 10 is a side view of a second embodiment of the device of the present disclosure having dispensed markers, including an RFID chip, into the sub-mucosa of the intestinal wall.

FIG. 11 is a view from the distal end of a colonoscope and RFID reader of the present disclosure.

FIG. 12 is a side view of the distal end of a colonoscope and RFID reader of the present disclosure.

FIG. 13 is a side view of the distal end of a colonoscope with antenna and RFID reader of the present disclosure.

FIGS. 14 A, 14B, 14C, and 14D show progressive views of the delivery of a detachable RFID marker in the tip of a needle-like structure to the spot of biopsy.

### DETAILED DESCRIPTION

The system and method disclosed herein can be used to mark a biopsy site or polyp resection site both simply and reproducibly, typically during colonoscopy, in a manner that allows for subsequent location of the site. In the broadest terms, one device described here is used to firmly grasp the polyp site or biopsy site. Once the tissue is held in place, a needle is advanced into the submucosal layer of the intestinal wall and a small quantity of radiopaque markers are introduced into this layer with or without a small amount of an ink-like stain, or dye. By placing these radio opaque markers into the submucosal layer of the intestine, these materials are protected and embedded more permanently.

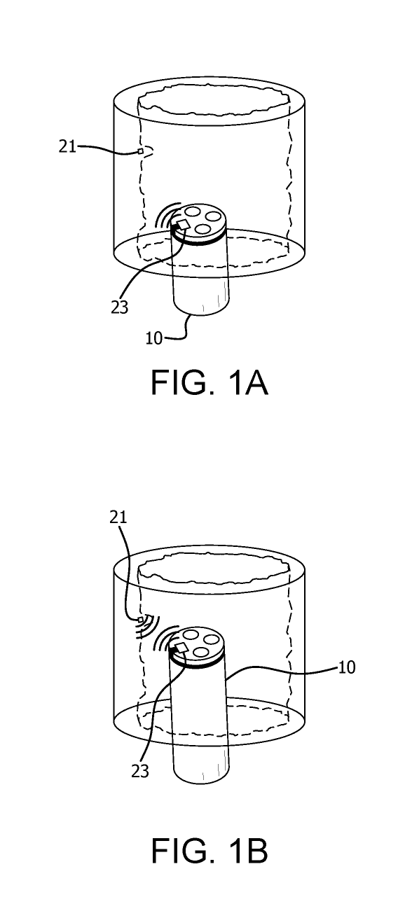

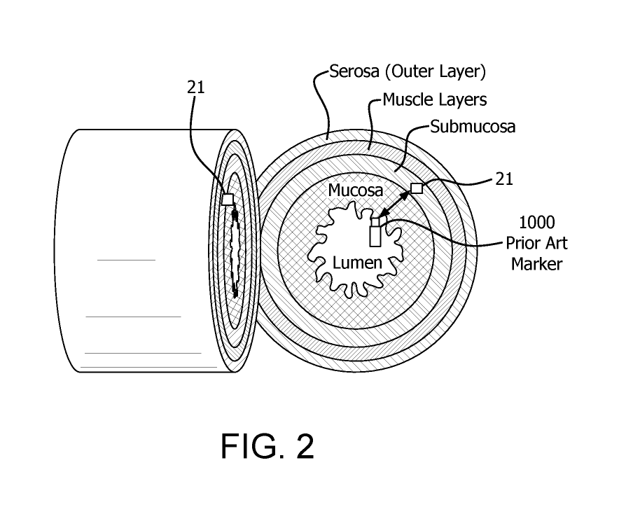

FIGS. 1A and 1B depict a series of steps in achieving one of the goals of the instant disclosure: to be able to locate accurately a biopsy site. To do that, a colonoscope 10 of the present disclosure is shown being advanced through the intestine in FIG. 1A. At the tip of colonoscope 10 is an RFID reader 23, shown with an antenna wrapping around the end of the scope. RFID reader 23 continually emits a signal that will excite the RFID marker chip 21 embedded in the intestinal wall. As the RFID reader 23 is advanced toward the marker chip 21 as shown in FIG. 1B, the signals emitted therefrom excite RFID chip 21, signaling its location to the reader 23. As an alternative, the chip can be replaced with a radiopaque marker such as a pellet, and can be located with an x-ray device such as fluoroscopy as will be described herein. FIG. 2 provides background anatomical information about the structure of the intestine and how that structure relates to the placement of markers such as marker 21 of the instant disclosure as shown therein. The lumen is the inner opening of the intestine in which the colonscope 10 of this disclosure may be inserted. Marker 1000 is the traditional clip marker of the prior art, which, when dispensed in accordance with prior art systems and methods, hangs down into the lumen of the intestine and is subject to movement as waste moves through the intestine. A marker 21 of the present disclosure is shown in FIG. 1B being placed in the submucosal layer of the intestine wall, out of the way of waste moving through the intestinal lumen, and otherwise less prone to dislodgment.

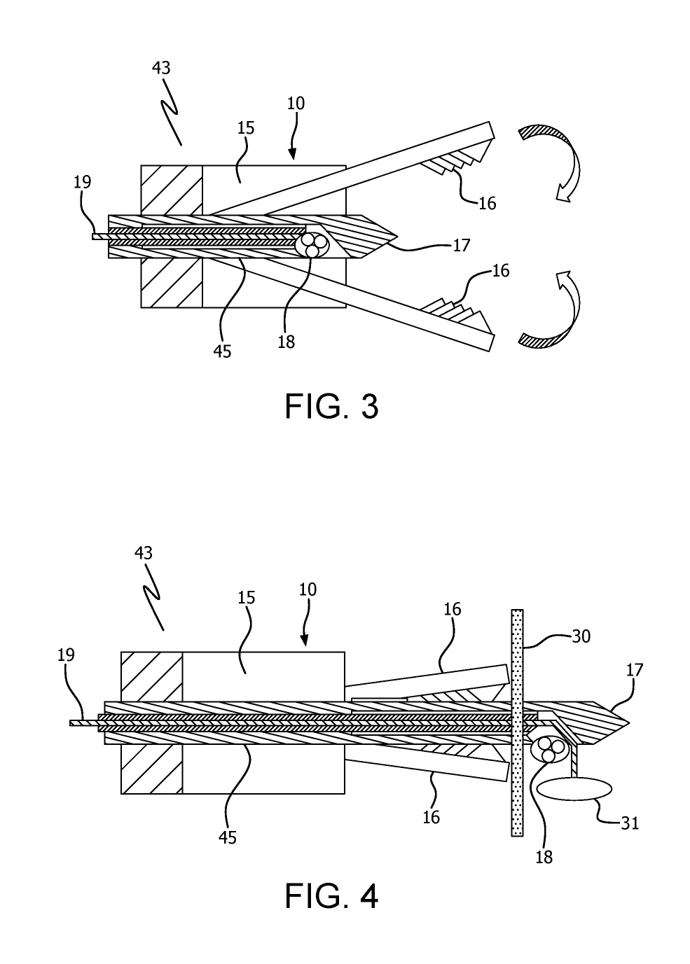

In one implementation of the instant disclosure, as shown in FIG. 3, disposition tool 45 of the instant disclosure is passed through an instrument port 15 of endoscope 10. Disposition tool 45 may include small jaws 16 to firmly grasp the inner wall of the intestine at the site, such as with a trigger type mechanism (not shown).

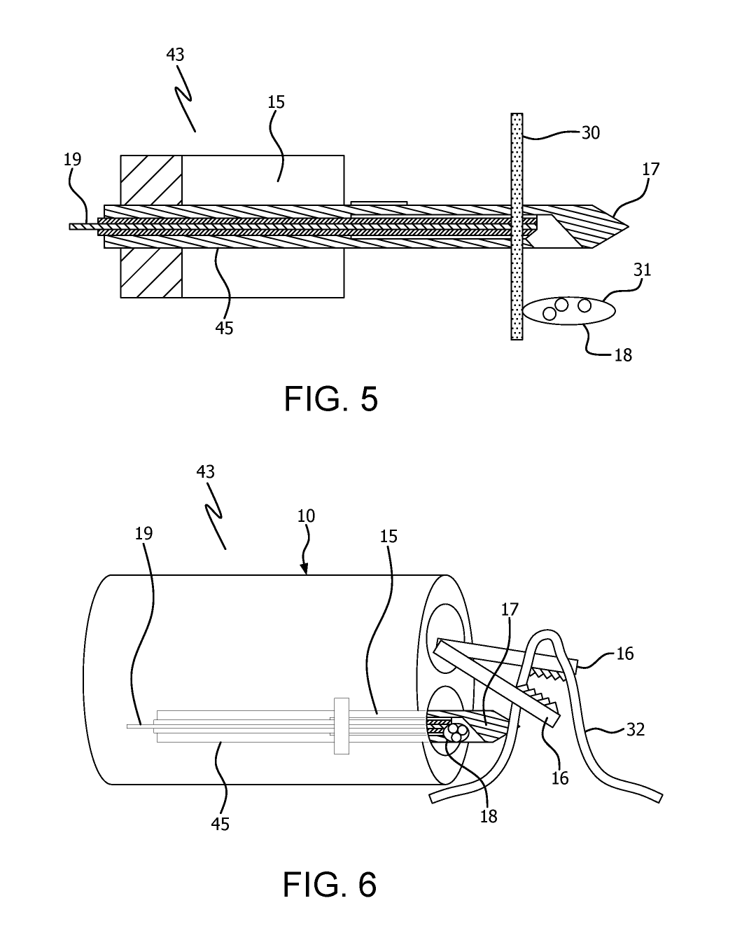

Once the soft tissue is held by jaws 16, it can be slightly elevated or otherwise manipulated or held in place as needed for subsequent operation of disposition tool 45. In one implementation, disposition tool 45 includes a needle 17, or another RFID marker delivery device, and a plunger or other suitable configuration to advance the needle 17. In the illustrated implementation, needle 17 is configured to hold RFID marker 21 in the form of microspheres 18 and a stain that is created by ink pumped through tube 19 within needle 17. Disposition tool 45 advances needle 17 into the submucosal layer 30 as shown in FIG. 3. Upon further actuation, as shown in FIG. 4, disposition tool 45 releases microspheres 18 and stain 31 into the proper layer 30, thereby protecting these markers from further disruption or injury. Once ink 31 and markers in the form of RFID beads 18 have been dispensed as shown in FIG. 5, the jaws 16 may be released to allow disposition tool 45 to be withdrawn.

In one possible variation, shown in FIG. 6, disposition tool 45 is configured and orientable endoscopically to raise polyp or other soft tissue sites 32 relative to the adjacent luminal wall portions, while simultaneously or sequentially locating needle 17 in a suitable position to dispose RFID markers 21 (such as in the form of beads 18) in the submucosa of the site 32. Although the more common configuration is to grasp and remove a polyp or soft tissue lesion of interest, and place a marker beneath that site, another option is simply to mark a site without retrieval of the polyp. In this case, a simple injection of saline into the wall of the intestine is performed at the site of interest. This saline injection elevates and separates the tissues within the wall of the intestine, allowing the needle to be advanced into this site and dispense its payload of markers.

The needle-like structure 45 of the instant invention is a standard 16 gauge needle, having a diameter of 1.651 mm, although a 14 gauge needle (with a diameter of 2.108 mm) can also be of practical use in the application disclosed.

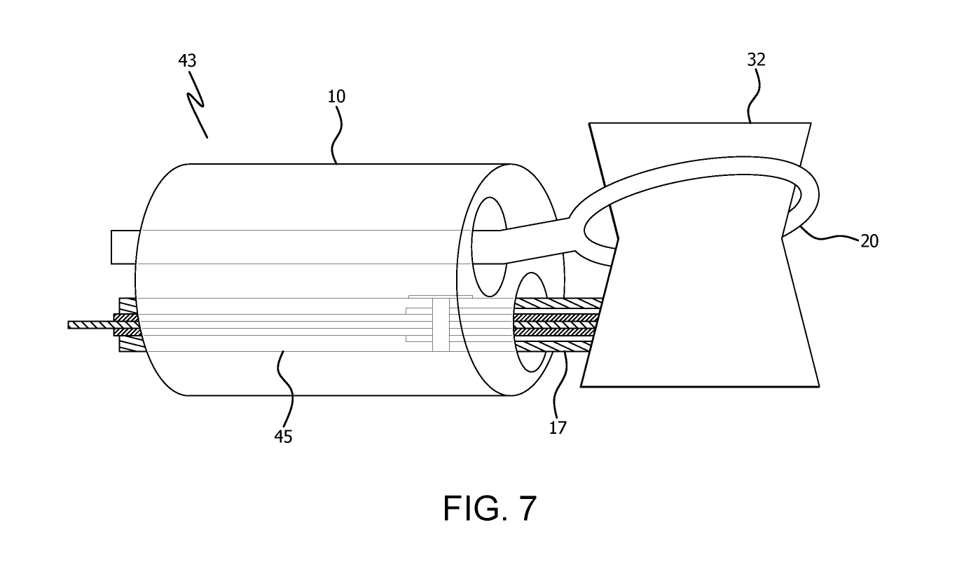

Another embodiment of this device involves a snare (or lasso) 20 in FIG. 7, that is placed around polyp 32 for removal. Once the polyp 32 is firmly grasped or snared, it is elevated in order to facilitate injection and placement of the microspheres 18 and ink 31 beneath it. The polyp 32 can then be detached from the intestinal wall 30 and the specimen can be withdrawn along with, or separately from the harvest/marking instrumentation. The stain 31 and microspheres 18 are left behind in the intestinal wall submucosa at the site of interest.

Sites of polyps or other soft tissue sites 32, once marked using the above-described features of system 43, may be readily located as described below so as to be resected or otherwise operated upon, regardless of whether such operations are performed endoscopically during the marking procedure, or subsequent thereto, whether endoscopically, laparoscopically, or otherwise. In the case of a colonoscopy, as just one possible method of this disclosure, colonoscope 10 is withdrawn after marking, and one or more marked polyp sites or intestinal sites are locatable from outside the intestine during a separate laparoscopic procedure.

The RFID markers 21 are thus useful for tracking and distinguishing multiple sites having a polyp or other tissue condition of interest, whether pre-removal or post-removal for examination pursuant to a biopsy. The RFID signals from RFID markers 21 are detectable from outside the lumen within which the markers are placed, thus facilitating subsequent laparoscopic or other non-endoscopic operations, both immediately after marking or in unrelated procedures in the future. Another variation of this device utilizes collagen or similar coating over microspheres 18 to facilitate their attachment within the intestinal wall.

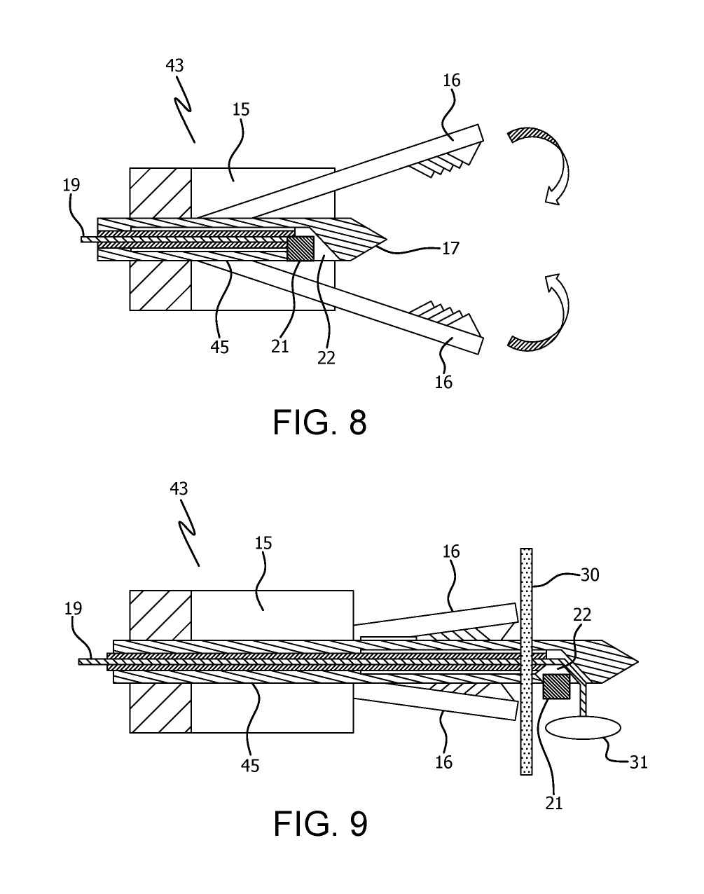

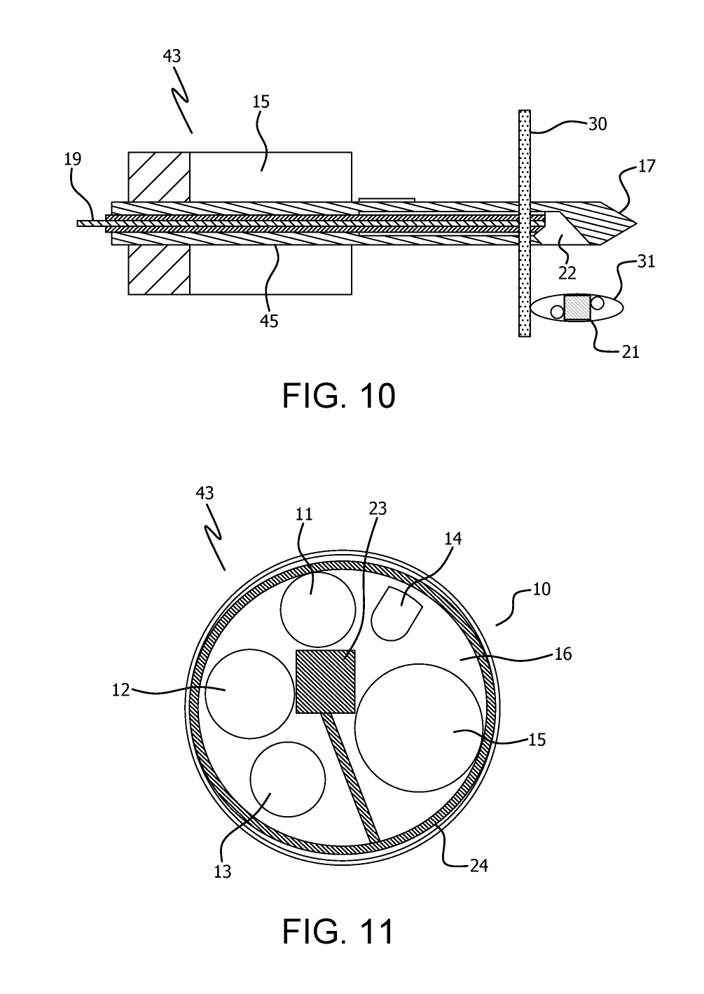

Another embodiment of the device of this disclosure is based on the insertion of an RFID chip 21 as shown in FIG. 8 into the wall of the intestine using a specialized needle 17 that incorporates a side port, or notch, 22 to deliver the chip 21 precisely into the therapeutically desired layer within the wall of the intestine and thereby with minimal risk of intestinal wall perforation. Said side port 22 is dimensioned so as to hold for placement up to three RFID marker chips 21, each of 1.5 mm in width and length, such that side port 22 has a width of at least 1.5 mm and longitudinally measures at least 4.5 mm. By placing the marker 21 within the tight confines of the wall of the intestine (intima media) 30 as shown in FIG. 8, the marker is less prone to move after placement. FIG. 9 also shows the infusion of stain 31 into the intestine wall 30 as a second marker, along with chip 21, for identifying the location of the biopsy, as such double marking result is shown in FIG. 10.

In one possible implementation of the present disclosure, the corresponding RFID chip 21 may be coded with a serial number unique to the corresponding polyp site. The serial number may be selected to be a simple two or three digit number, or other identifying indicia, so that the marker location of each polyp within a patient is unique, while remaining sufficiently generalized between patients so as to be consistent with any applicable ethical or regulatory frameworks.

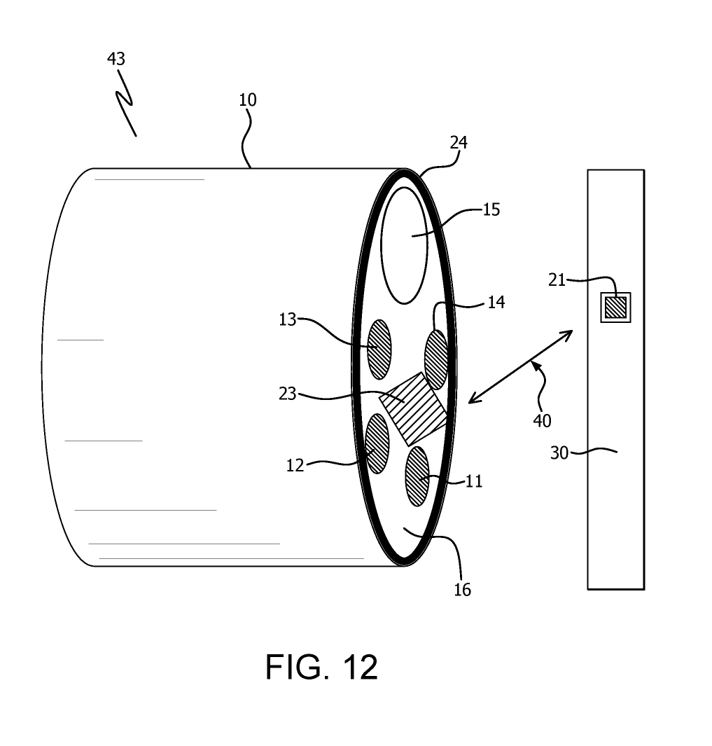

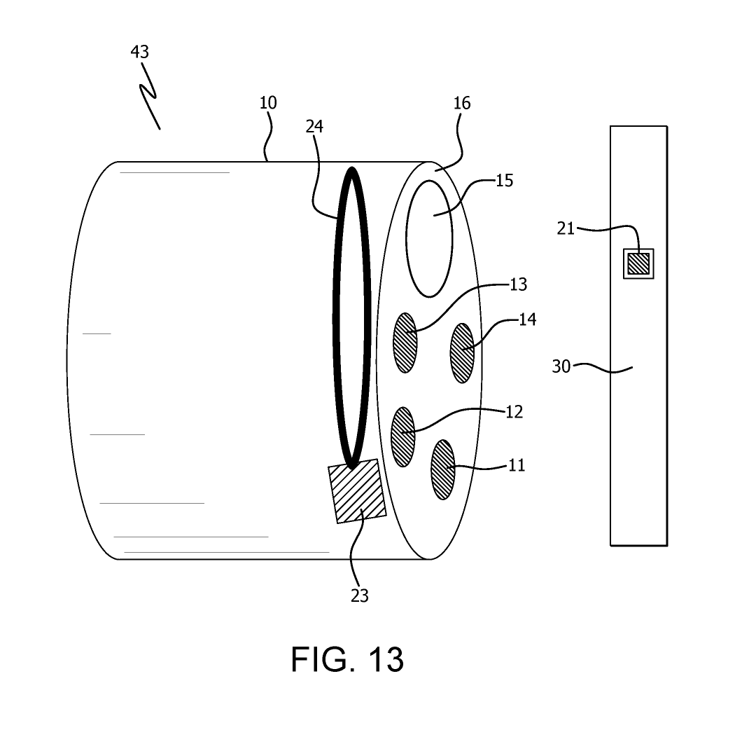

As shown in FIG. 11, an endoscope 10 can be outfitted with an RFID reader 23 and its accompanying antenna 24 that can be used to find and identify a marker such as RFID chip 21 as lodged in the intestine wall 30 of FIG. 9. In FIG. 12, RFID chip 21 is identifiable by RFID reader 23 and its antenna 24 over a distance identified as 40 after reader 23 and antenna 24 have been inserted through an endoscope or through a laparoscope, or through endoscope 10 as shown in FIG. 1A. Distance 40 is on the order of zero to five or six centimeters. FIG. 13 shows another embodiment in which an RFID reader 23 and its antenna 24 are located on the outside wall of endoscope 10 which thereby occupies less area on the distal end 16 of scope 10. Additionally, an RFID chip 21 can be constructed so as to be radiopaque, so that it could also be located with X-ray devices without intrusion into the body.

Implantation of RFID chip 21 may be accomplished without the need for a specially designed needle, such as the case where the chip is so small that it can fit through the lumen of a regular needle. In this case, a single or group of chips could be simply injected into the wall of the intestine. In this embodiment, the necessary radiopacity could be achieved by injecting several RFID chips simultaneously. In order to decrease its size, RFID chip may be a passive (i.e., non-powered) chip instead of an active marker that requires a power source. Additionally, the RFID chip, in other implementations, may also be coated with collagen in order to facilitate embedding the marker in the intestinal submucosa.

In another implementation, the RFID chip 21 is in the form of a passive RFID tag to be placed at the biopsy site by disposition tool 45 in the form of a needle 17, and a corresponding reader 23 sized to be removably insertable through an endoscope 10.

RFID reader 23 as shown in FIG. 11 is small enough to fit through standard endoscope ports. RFID reader 23 includes a suitable processor or programming to use a proprietary wavelength for detecting and reading codes, so that unauthorized readers would not be effective in accessing or activating RFID chip 21 or information stored therein.

RFID chips 21 in another embodiment as shown in FIG. 10 are delivered simultaneously with radiopaque beads, so that if for some reason the RFID could not be found electronically, the site could still be located by x-ray equipment. RFID reader 23 in one embodiment, may be configured to operate when its antenna or other signal receiver is completely or partially located outside the body. As in FIG. 12, RFID markers 21 and associated antennae 24 are no larger than required to detect signals at distance 40 which has a maximum, predetermined value based on required strength of signals from all possible locations where markers would be disposed in a lumen, or as also covered hereby in other internal anatomical locations associated with the corresponding medical procedures. In the invention of this disclosure applied to colonoscopy, for example, RFID markers 21 have characteristics to enable their placement at any location of the gastrointestinal tract to be detected by RFID reader 23 from outside the tract or associated lumen, not only to enable laparoscopic procedures, but from a sufficient distance to minimize the extent of follow-on invasive surgical procedures to locate the markers. In one suitable application, such maximum potential distance is 5 to 6 cm

In the implementation shown in FIG. 11, RFID reader 23 is configured to include antenna wires extending through the port of an endoscope 10. In another implementation RFID reader 23 can be inserted laparoscopically and extended into the region of the marker to find and identify the desired RFID chip. RFID reader 23 can also be constructed as a device sized and otherwise configured to reside within endoscope 10 or to attach to the end of endoscope 10.

In FIG. 12 the implementation includes RFID chips 21 that are of the passive type, and a reader 23 housed on the tip of endoscope 10 of system 43, which, thus configured, detects signals by passing antenna 24 of reader 23 within a few inches (5 or 6 centimeters) of a corresponding one or more of RFID chips 21. In juxtaposition to the manner of FIG. 12, FIG. 13 shows antenna 24 attached to the outer edge of endoscope 10 rather than encircling its distal end. As shown in the progressive drawings in FIGS. 1A and 1B, detection by system 43 may occur upon movement of RFID reader 23 toward marker 21, either by inductively or capacitively exciting RFID chip marker 21 when in the proximity required by the strength of signal, which chip 21 then relays a signal back to reader 23.

System 43 of FIG. 10 is yet another implementation that includes one or more processors with suitable programming for storing the above-described serial numbers of RFID markers 21 or other related information associated with RFID marking procedures. In this way, additional personalized data can be recorded on marker 21 which could include date of implantation, and any other pertinent medical or personal data as needed.

Additional programming of system 43 may likewise be provided for processing signal detection by RFID reader 23. In the version of FIG. 10, the encoded marker 21 data can be programmed to limit access to a specific reader. This can be accomplished by using proprietary wavelengths and/or encryption protocols.

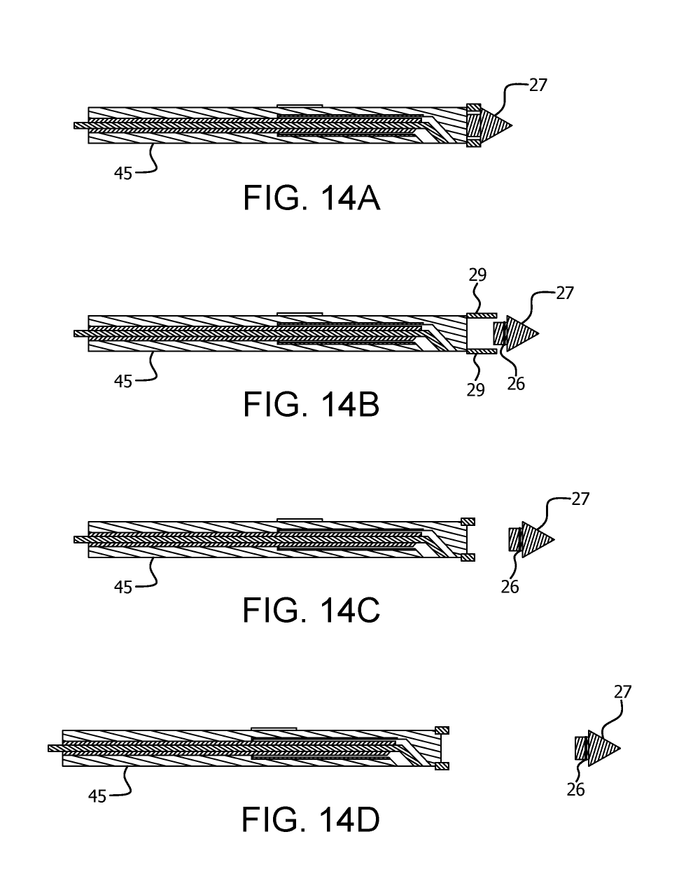

FIG. 14A depicts an implementation using a specialized needle-like structure 45 for which the RFID marker 27, formed as the tip of an arrow, is detachably positioned at the leading distal end of structure 45. As shown in the progressive figures on this page of drawings, RFID marker 27 is detached by the physician as shown in FIG. 14B by use of trigger means well known in the art and propelled with momentum as parallel segments 29 are triggered outward from structure 45 toward the submucosa of the biopsy site. As shown in the next progressive drawing of FIG. 14C, arrow-shaped RFID marker 27 and its attached antenna 26 shown from a side view of the trailing segment of marker 27 have been detached fully from structure 45. In next progressive drawing FIG. 14D, marker 27 and its antenna 26 are shown closing in on the applicable biopsy site having momentum to lodge said marker and antenna combination in the submucosa at that site.

It should be appreciated that the instant disclosure is not to be seen as limited to marking and detection of sites in the colon, much less in the entire gastrointestinal tract by use of endoscopy. Using the RFID technology described herein, a chip can be implanted in a variety of tissues within the gastrointestinal tract as well as in many tissues in the human body, including, but not limited to, marking of tissue in patients having Barrett's esophagus and tagging of bleeding lesions in the liver.

What is claimed is:

1. A system for marking a location of removed tissue comprising:
a scope having a port for a biopsy instrument;
a biopsy instrument housed within said port;
a disposition tool housed within said biopsy instrument; and
a marker consisting of at least one selected from the group of a radio-frequency identification capable chip, a radiopaque marker, radiopaque material, and radio-frequency identification material,
wherein the disposition tool is configured to carry the marker, deliver the marker to the location of removed tissue, and dispose the marker within tissue remaining at the location.

2. The system of claim 1 in which the scope is selected from the group comprised of colonoscopes and endoscopes.

3. The system of claim 2, wherein the scope is a colonoscope, wherein the marker is a passive radio-frequency identification capable chip, and wherein the system further comprises an RFID reader attached to the distal end of said colonoscope.

4. The system of claim 3, further comprising dye, and wherein the disposition tool is configured to deliver the dye at the time of disposition of the marker.

5. The system of claim 1 in which the composition of a radiopaque marker shall be selected from the group comprised of surgical grade steel, titanium, and metallic composites.

6. The system of claim 1 in which the radiopaque material is selected from the group comprised of radiopaque microspheres, radiopaque microspheres coated with collagen, and radiopaque microspheres having radio-frequency identification capability.

7. The system of claim 1 in which the radio-frequency identification material is microspheres having radio-frequency identification capability.

8. The system of claim 4 in which at least one marker is a radio-frequency capable identification chip.

9. The system of claim 8 in which the radio-frequency capable identification chip is selected from the group comprised of passive RFID chips and active RFID chips.

10. The system of claim 9 in which the radio-frequency identification capable chip is radiopaque.

11. The system of claim 1 in which the disposition tool is a needle-like construct.

12. The system of claim 11 in which the needle-like construct is a specialized needle having a side port configured for disposition of at least one radio-frequency capable identification chip.

13. The system of claim 1 in which the biopsy instrument is selected from a group comprised of an instrument with two sawtooth jaws, an instrument having a lasso, and an instrument having a snare.

14. A method for marking sites of removed tissues comprising:
performing a biopsy at a site on a tissue using an instrument;
passing a disposition tool through said instrument; and
using said disposition tool to dispose at said site a marker selected from the group comprised of a radio-frequency identification capable chip, a radiopaque marker, radiopaque material, and radio-frequency identification material.

15. The method of claim 14 comprising the additional step of:
using said disposition tool to dispose at said site at least one additional marker, selected from the group comprised of dye, radiopaque material, and radio-frequency identification material.

16. A method for locating a site of biopsy on a tissue after the performance of the biopsy using a biopsy instrument at the biopsy site on the tissue comprising;
passing a disposition tool through said biopsy instrument;
using said disposition tool to dispose a marker at said site, which marker is selected from the group comprised of a radio-frequency identification capable chip and microspheres having radio-frequency identification capability;
passing a radio-frequency identification reader in proximity to said marker; and
locating said biopsy site with said reader.

17. A method for marking sites of biopsied tissues and subsequently locating such sites of such biopsies on such tissues comprising:
performing a biopsy at a site on a tissue using an instrument;
passing a disposition tool through said instrument;
using said disposition tool to dispose a marker at said site which marker is selected from the group comprised of a radio-frequency identification capable chip and microspheres having radio-frequency identification capability; and
passing a radio-frequency identification reader in proximity to said marker; and
locating said site with said reader.

18. The method of claim 17 comprising the additional step of:
using said disposition tool to dispose at least one additional marker at said site selected from the group comprised of a radio-frequency identification capable chip and microspheres having radio-frequency capability.

19. In an endoscope having a port for an instrument for performing a biopsy at a site in the intestine of a patient, the improvement comprising:
an instrument for performing a biopsy at a site in the intestine of a patient housed within said port having a disposition tool housed therein; and
a marker in the form of a radio-frequency identification capable chip attached to said tool,
whereby said marker when disposed from said disposition tool becomes located in the intestine at the site of biopsy performed by said instrument.

20. The improvement of claim 19 further comprising at least one additional marker selected from the group comprised of a radiopaque marker, dye, radiopaque material, and radio-frequency identification material.

21. The improvement of claim 21 in which the composition of a radiopaque marker shall be selected from the group comprised of surgical grade steel, titanium, and metallic composites.

22. The improvement of claim 20 in which the radiopaque material is selected from the group comprised of radiopaque microspheres, radiopaque microspheres coated with collagen, and radiopaque microspheres having radio-frequency identification capability.

23. The improvement of claim 19 in which the radio-frequency identification capable chip is selected from the group comprised of passive RFID chips and active RFID chips.

24. The improvement of claim 20 in which the radio-frequency identification material is microspheres having radio-frequency identification capability.

26. The improvement of claim 17 in which the disposition tool is a needle-like construct.

27. The improvement of claim 26 in which the needle-like construct is a specialized needle having a side port configured for disposition of an RFID chip.

28. The improvement of claim 19 in which the instrument for performing a biopsy is selected from a group comprised of an instrument with two sawtooth jaws, an instrument having a lasso, and an instrument having a snare.

29. The improvement of claim 19 further comprised of a radio-frequency identification reader mounted on said endoscope.

30. In an endoscope, the improvement comprising a radio-frequency identification reader mounted on said endoscope.

31. An article of manufacture that is a needle having a side port configured for disposition of at least one radio-frequency identification capable chip.

32. The article of manufacture of claim 31 in which a radio-frequency identification capable chip is selected from the group comprised of active RFID chips and passive RFID chips.
